(19) United States
(12) Patent Application Publication
(10) Pub. No.: US 2008/0188510 A1
(43) Pub. Date: Aug. 7, 2008

(54) NOVEL METHODS USING ZONISAMIDE

(75) Inventor: Hiroshi Yoshino, Tsukuba-shi (JP)

Correspondence Address: DARBY & DARBY P.C., P.O. BOX 770, Church Street Station, New York, NY, 10008-0770, US

(73) Assignee: Eisai R & D Management Co., Ltd., Bunkyo-ku (JP)

(21) Appl. No.: 11/419,816

(22) Filed: May 23, 2006

Related U.S. Application Data
(60) Provisional application No. 60/684,150, filed on May 23, 2005.

(51) Int. Cl. A61K 31/423; A61P 25/00; A61K 31/445; A61K 31/4355

(52) U.S. Cl. 514/288; 514/379; 540/576

(57) ABSTRACT

The present invention relates to usefulness of zonisamide as a pharmaceutical composition for treating dementia or cognitive impairment.

The present application claims the benefit of U.S. provisional patent application No. 60/684,150 filed May 23, 2005, whose specification and claims are incorporated herein by reference.

### FIELD OF THE INVENTION

The present invention relates to usefulness of zonisamide as a pharmaceutical composition for treating dementia or cognitive impairment.

### BACKGROUND OF THE INVENTION

Dementias, especially Alzheimer's disease (AD) and senile dementia of the Alzheimer's type (SDAT) are diseases observed in various ethnic groups and races around the world. As the life expectancy of human is increasing, the number of aged population as well as the number of dementia patients is increasing as well. Therefore, increase in the number of dementia patients is becoming one of the recent social issues, and there has been a strong social demand for therapeutic agent development for these diseases. AD and SDAT are diseases that progressively present symptoms of dementia. The AD and SDAT patients are known to show change or decreased function of brain cholinergic nervous system (i.e., decrease in brain acetylcholine) in the basal forebrain that plays an important role in cognitive functions including memory. The amyloid hypothesis that stresses on the abnormal accumulation of amyloid β protein (Aβ) which is one of the major components of senile plaque, and the tau hypothesis that stresses on formation of neurofibrillary tangles caused by abnormal phosphorylation of tau and else are proposed as the causes of AD and SDAT.

Today, researches are conducted from every direction for understanding the mechanisms of onsets of such diseases as well as for developing therapeutic agents for such diseases. For example, in order to increase the amount of brain acetylcholine, a compound that inhibits activity of cholinesterase (ChE), i.e., an acetylcholine-degrading enzyme, and a compound that activates acetylcholine transferase (ChAT), i.e., an acetylcholine synthetase have been proposed. Muscarinic acetylcholine receptor agonist, NMDA (N-methyl-D-aspartate) receptor antagonist, AMPA (α-amino-3-hydroxy-5-methyl-4-isoxazole propionic acid) receptor antagonist and else have been proposed to act on receptors. Based on the amyloid hypothesis, inhibitors of Aβ production, inhibitors of Aβ aggregation, stimulators of Aβ degradation/elimination and else have also been proposed. In fact, a cholinesterase inhibitor (ChEI) is effective in treating patients with AD or SDAT. In particular, donepezil hydrochloride, an acetylcholinesterase inhibitor (AChEI) increases brain acetylcholine and is extensively used as a therapeutic agent for AD and SDAT (Japanese Patent No. 2578475).

On the other hand, zonisamide [chemical name: 3-sulfamoylmethyl-1,2-benzisoxazole and 1,2-benzisoxazole-3-methanesulfonamide; e.g., Merck Index, 12th Ed., 10323 (1996)] is disclosed for its usefulness as an antiepileptic drug for treating various epilepsy seizures (Japanese Examined Application No. 60-33114, Japanese Examined Application No. 61-59288, U.S. Pat. No. 4,172,896, Epilepsy Research 29, 109-114, 1998), for treating ischemic brain disorder (Japanese Examined Application No. 7-84384, U.S. Pat. No. 5,128,354, Brain Research 770, 115-122, 1997), and for treating diseases caused by neurodegeneration such as Parkinson's disease, Huntington's disease, chorea syndrome and distonia syndrome for it shows extremely strong suppressant action to dopaminergic neurodegeneration induced by MPTP (1-methyl-4-phenyl-1,2,3,6-tetrahydropyridine) (Japanese Patent No. 3364481, Neurosci. Res., 41, 397-399, 2001, Current Pharmaceutical Design, 10, 687-693, 2004). However, there is no teaching about the drug efficacy of zonisamide on dementia, especially on AD and SDAT.

### SUMMARY OF THE INVENTION

A novel improved method for treating diseases, disorders and syndromes characterized by conditions of dementia and/or cognitive impairment is required in this technical field. The present invention was completed by addressing these problems.

The present invention provides a therapeutic agent for dementia (e.g., AD, SDAT) or cognitive impairment, comprising zonisamide, a salt thereof, a solvate thereof or a prodrug thereof.

In another aspect, the present invention provides a use of zonisamide, a salt thereof, a solvate thereof or a prodrug thereof for producing a therapeutic agent for dementia (e.g., AD, SDAT) or cognitive impairment.

In another aspect, the present invention provides a method for treating dementia (e.g., AD, SDAT) or cognitive impairment, wherein an effective amount of zonisamide, a salt thereof, a solvate thereof or a prodrug thereof is administered to a patient in need of treatment for these diseases.

In yet another aspect, the present invention provides a pharmaceutical composition comprising zonisamide, a salt thereof, a solvate thereof or a prodrug thereof together with at least one selected from the group consisting of cholinesterase inhibitors, NMDA receptor antagonists and AMPA receptor antagonists.

The present invention also provides a method for treating dementia or cognitive impairment, comprising administering zonisamide, a salt thereof, a solvate thereof or a prodrug thereof together with at least one selected from the group consisting of cholinesterase inhibitors, NMDA receptor antagonists and AMPA receptor antagonists to a patient in need of treatment for these diseases.

According to a preferred aspect of the method of the invention, an effective amount of zonisamide, a salt thereof, a solvate thereof or a prodrug thereof and an effective amount of a therapeutic agent for dementia (e.g., AD, SDAT) or cognitive impairment are administered separately or as a pharmaceutical composition containing them together (a blended agent) to the patient.

### DETAILED DESCRIPTION OF THE INVENTION

Hereinafter, the present invention will be described in more detail. The following embodiments are examples for illustrating the present invention, while the present invention is not intended to be limited to these embodiments. All technical terms, scientific terms and terms of art used herein are used in the same sense as those generally understood by those skilled in the art to which the present invention pertains, and are used to simply illustrate a certain aspect and not to impose a limitation. Although preferred methods and materials are referable hereinbelow, similar or equal methods and materials as those described herein may be used for carrying out or testing the present invention. The present invention may be carried out in various embodiments without departing from the scope of the invention.

All of the prior art publication, laid-open patent applications, patents and other patent publications are incorporated herein by reference and may be used for carrying out the present invention.

1. Definitions

As used herein, the term “dementia” is used in the same meaning as the “mental deterioration.

The term “cognitive impairment” means acquired deficiency that interferes with independently functioning abilities of an individual. Examples of such deficiencies include one or more of the following: disturbances of memory function, deterioration or loss of problem-solving skill, disorientation and/or abstraction.

The term “dementia” refers to symptoms associated with overall deterioration of intellectual functions with clear consciousness, which is characterized by one or more of the following: disorientation, impaired memory, impaired judgment and impaired intellect. The symptoms of “dementia” are generally more serious than those of “cognitive impairment”, and may comprise symptoms of cognitive impairment.

Herein, the phrase “dementia/cognitive impairment” or “cognitive impairment/dementia” refers to both or either one of dementia and cognitive impairment. Diseases, disorders and syndromes characterized by symptoms of dementia and/or cognitive impairment are exemplified below.
(a) Alzheimer's disease, senile dementia of the Alzheimer's type, dementia associated with vascular disease, dementia caused by vascular disease, dementia associated with Parkinson's disease, dementia associated with Huntington's disease, dementia associated with alcohol addiction, Lewy body dementia and AIDS dementia;
(b) mild cognitive impairment, age-related impaired memory;
(c) dementia/cognitive impairment associated with epilepsy, dementia/cognitive impairment associated with cerebral tumor, dementia/cognitive impairment associated with brain injury, dementia/cognitive impairment associated with multiple sclerosis, dementia/cognitive impairment associated with Down's syndrome, dementia/cognitive impairment associated with Rett's syndrome, dementia/cognitive impairment associated with progressive supranuclear palsy, dementia/cognitive impairment associated with frontal lobe syndrome and dementia/cognitive impairment associated with neurologic and/or psychiatric conditiones (including schizophrenia and associated mental disorders); and
(d) cognitive impairment associated with traumatic brain injury, cognitive impairment associated with aftereffects of coronary-artery bypass surgery, cognitive impairment associated with electric shock therapy and cognitive impairment associated with chemotherapy.

The term “patient” refers to an animal, preferably a mammal and includes a human patient suffering from dementia/cognitive impairment described above.

The term “mammal” means any animal that is classified as a mammal, including human or non-human mammals (e.g., mouse, rat, hamster, guinea pig, rabbit, pig, dog, horse, cow, monkey, etc.). Preferably, a mammal described herein is human. In this case, the term “patients” includes adults and children and both males and females. Children include newborn infants, infants and adolescents.

“Alzheimer's disease” (AD) and “senile dementia of the Alzheimer's type” (SDAT) are diseases that induce deterioration of cognitive function, altered personality and else by atrophy of brain tissue and appearance of senile plaque in cerebral cortex. Symptoms of AD and SDAT include progressive disorientation, amnesia and aphasia, which eventually cause incompetence, speech loss and akinesia. Pathological signs of AD and SDAT include, for example, presences of neurofibrillary tangle, senile plaque and amyloid vascular disorder. Since there is no essential difference between these diseases, they are collectively called Alzheimer-type dementia or AD, where presenile onset at the age below 65 is called AD or early-onset type and onset at the age of 65 or older is called SDAT or late-onset type. In a preferred aspect, the present invention provides a method for treating AD or SDAT, comprising administering an effective amount of zonisamide, a salt thereof, a solvate thereof or a prodrug thereof to a patient.

“Dementia associated with vascular disease” and “dementia caused by vascular disease” are also called vascular dementias and generally refer to dementia that occurs because of cerebrovascular disease (e.g., infarction of the cerebral hemispheres). Generally, this dementia follows a fluctuating course with periods of improvement and stepwise deterioration. “Vascular dementia” can include one or more of symptoms of disorientation, impaired memory and impaired judgment. Early markers for vascular dementia can include urinary dysfunction and/or gait disturbances. Vascular dementia can be caused by discrete multiple infarctions. It may also develop because of, for example: autoimmune vasculitis that is a condition observed in systemic erythematosus; infectious vasculitis such as Lyme's disease; recurrent intracerebral hemorrhage; and/or other vascular causes including stroke. “Vascular dementia” is sometimes called cerebrovascular dementia (VD; Vascular Dementia). According to a preferred embodiment, the present invention provides a method for treating dementia defined herein that is associated with or caused by a vascular disease.

“Parkinson's disease” is neurological syndrome usually resulting from the neurotransmitter dopamine as the consequence of degenerative or vascular or inflammatory changes in the basal ganglia, and is characterized by rhythmic muscular tremor, rigidity of movement, Destination, droopy posture and/or masklike facies. In a preferred aspect, the present invention provides a method for treating dementia defined herein that is associated with or caused by Parkinson's disease.

“Huntington's disease” is an autosomal dominant genetic disease characterized by dancing-like movement and progressively reduced intelligence and is an inherited disease associated with a decrease in the number of brain neurons in the brain basal ganglia, which act to keep the balance of the body or to regulate motor function. At the early stage of the disease, abnormal movements such as frown, sticking out tongue and dancing-like movements of moving and stopping arms and legs and a change in mental state such as easily becoming bad-tempered are observed. As the stage of disease progresses, the power of memory weakens and dementia occurs. In a preferred aspect, the present invention provides a method for treating dementia defined herein that is associated with or caused by Huntington's disease.

The term “alcohol addiction” comprises alcohol dependency. Alcohol dependency refers to psychiatric conditiones caused by alcohol intake and accompanying physical conditions, showing other behavioral reactions such as those associated with continuous or periodical compulsive desire of drinking, and is characterized by loss of drinking control and disturbance in social and carrier functions. Patients showing alcohol dependency are also known to show drop out of frontal lobe neurons, rapid progression of atrophy of brain as compared to similarly-aged human and malnutrition such as shortage of vitamin B1 due to poor dietary intake for too much drinking, and likely to result in impaired memory and disorientation. The present invention provides a method for treating dementia/cognitive impairment defined herein that is associated with or caused by alcohol addiction.

“Lewy body dementia” is characterized by one or more symptoms of the following: occurrence of dementia that has overlapping features with Alzheimer's disease; appearance of features of Parkinson's disease; and early occurrence of hallucination. In general, Lewy body dementia is characterized by daily fluctuation of severity of the symptom. The disease is named for the presence of abnormal deposits in neurons called Lewy bodies.

“AIDS dementia” is caused by complication associated with HIV disease, namely AIDS. Symptoms associated with AIDS dementia may include one or more of the following symptoms: headache, postorbital pain, photophobia, depression, mania, irritability, mental disorder, mental retardation, carelessness, blunted affect, weak concentration, poor memory, motor abnormality, gait abnormality (motor incoordination), altered personality, disorientation, impaired memory, impaired judgment and impaired intellect.

According to a preferred embodiment, the present invention provides a method for treating dementia/cognitive impairment defined herein that is associated with or caused by AIDS.

“Mild cognitive impairment” refers to one or more of milder symptoms of disorientation, impaired memory, impaired judgment and/or impaired intellect. Although elderly persons often suffer from mild cognitive impairment, generally impaired memory, this is below the level to be diagnosed as AD.

“Age-related impaired memory” is characterized by impaired memory with age that can be seen in elderly persons.

In a preferred aspect, the present invention provides a method for treating dementia/cognitive impairment defined herein by administering zonisamide described herein, a salt thereof, a solvate thereof or a prodrug thereof to a patient, preferably a patient suffering from age-related impaired memory or mild cognitive impairment.

“Dementia/cognitive impairment associated with epilepsy” refers to dementia/cognitive impairment defined herein that is associated with or caused by epilepsy. Zonisamide, a salt thereof, a solvate thereof or a prodrug thereof described herein is also useful in a method for treating side-effects (e.g., dementia/cognitive impairments) caused by drugs used for treating epilepsy. In a preferred aspect, the present invention provides a method for treating dementia/cognitive impairment defined herein that is associated with or caused by epilepsy.

“Cognitive impairment associated with cerebral tumor” refers to dementia/cognitive impairment defined herein that is associated with or caused by cerebral tumor. In a preferred aspect, the present invention provides a method for treating dementia/cognitive impairment defined herein that is associated with or caused by cerebral tumor.

“Dementia/cognitive impairment associated with brain injury” refers to dementia/cognitive impairment defined herein that is caused by or associated with brain injury or brain inflammatory disease. In a preferred aspect, the present invention provides a method for treating dementia/cognitive impairment defined herein that is associated with or caused by brain injury.

“Multiple sclerosis” is a disease induced by appearance of plaque (e.g., blob) in brain and spinal cord, and is characterized by a certain degree of palsy, tremor, nystagmus and/or speech disability. The symptoms of multiple sclerosis depend on the location of lesion in the brain. Preferably, the present invention provides a method for treating dementia/cognitive impairment defined herein that is associated with or caused by multiple sclerosis.

“Down's syndrome” is a disease that is caused due to chromosome 21 (or a critical portion thereof) being a triploid instead of a diploid in some or all of the cells, and is mental retardation syndrome related to many abnormal conditions. In a preferred aspect, the present invention provides a method for treating dementia/cognitive impairment defined herein that is associated with or caused by Down's syndrome.

“Rett's syndrome” namely hyperammonemia associated with brain atrophy is progressive syndrome characterized by autism, dementia, cognitive impairment, motor incoordination and/or symptoms such as purposeless hand moves. In a preferred aspect, the present invention provides a method for treating dementia/cognitive impairment defined herein that is associated with or caused by Rett's syndrome.

“Progressive supranuclear palsy” is also known as Steele-Richardson-Olszewski syndrome, which is a rare brain disorder characterized by abnormal control of gait and/or balance. The most obvious sign of the disease is inability of appropriately directing the eyes to the object, which results from lesion in an area of the brain for regulating ocular movement. Other symptoms of progressive supranuclear palsy include altered mood and behavior (e.g., depression, blunted affect, cognitive impairment and/or progressive mild dementia). In a preferred aspect, the present invention provides a method for treating dementia/cognitive impairment defined herein that is associated with or caused by progressive supranuclear palsy.

“Frontal lobe syndrome” is a disease resulting from various causes including one or more of stroke, head injury, multiple infarct dementia, frontal lobe tumor and postencephalitic syndrome. Symptoms of frontal lobe syndrome include one or more of the following: mood lability, decrease or loss of judgment and insignt, inappropriate or disinhibition behavior, impaired memory, decrease in attention span, inability to shift set of thinking, difficulty in planning and practicing, and mobility or sensory impairments specific to other areas in brain suspected of simultaneous disorders. In a preferred aspect, the present invention provides a method for treating dementia/cognitive impairment defined herein that is associated with or caused by frontal lobe syndrome.

“Dementia/cognitive impairment associated with neurologic and/or psychiatric conditiones” includes schizophrenia and associated psychiatric/psychological disorders (associated mental disorders).

“Schizophrenia” is a psychosis characterized by disorder in the thinking processes, such as delusions and hallucinations, and excessive withdrawal of the patient's interest from other people and the outside world, and the investment of it in his own.

Patients diagnosed with schizophrenia are often associated with dementia/cognitive impairments caused by advance of the underlying disease and/or as a side-effect of treatments with an antipsychotic drug. In a preferred aspect, the present invention provides a method for treating dementia/cognitive impairments defined herein that are associated with or caused by schizophrenia and related psychiatric/psychological disorders (e.g., including schizoaffective disorders). In another aspect, the present invention provides a method for treating dementia/cognitive impairments, as defined herein that is a side-effect of an antipsychotic drug. As used herein, the term “schizophrenia” refers to process-reactive schizophrenias, which includes, for example, chronic schizophrenia, ambulatory schizophrenia, catatonic schizophrenia, disorganized schizophrenia, latent schizophrenia, paranoid schizophrenia, pseudoneurotic schizophrenia, residual schizophrenia, and simple schizophrenia.

“Cognitive impairment associated with traumatic brain injury” refers to a cognitive impairment defined herein that is associated with or caused by traumatic brain injury, including aftereffects of head injury and other head injuries such as accidental injury and/or sports injury. “Cognitive impairment caused by traumatic brain injury” includes boxer's dementia, which is a severe brain injury caused by repetitive beating on head (e.g., by boxing). Boxer's dementia is chronic and progressive clinical syndrome characterized by neurologic signs associated with at least one sign/symptom selected from the group consisting of mental disorder, dementia, altered personality, social dysfunction and parkinsonian syndrome, specifically, neurologic signs of injuries on pyramidal tract, extrapyramidal tract and cerebellar system. Examples of signs of parkinsonian syndrome include tremor, dysarthria, rigidity, bradykinesia and other extrapyramidal tract signs.

In a preferred aspect, the present invention provides a method for treating cognitive impairment defined herein that is associated with or caused by traumatic brain injury.

“Cognitive impairment associated with aftereffects of coronary-artery bypass surgery” refers to cognitive impairment defined herein that is caused by or associated with aftereffects of coronary-artery bypass surgery or ischemic vascular disease. In a preferred aspect, the present invention provides a method for treating cognitive impairment defined herein that is associated with or caused by aftereffects of coronary-artery bypass surgery.

“Cognitive impairment associated with electric shock therapy” refers to cognitive impairment defined herein that is caused by or associated with electric shock therapy (e.g., electric shock therapy for depression, catatonia, Parkinson's disease or chronic pain symptom). In other aspect, the present invention provides a method for alleviating (e.g., reducing or eliminating) cognitive impairment caused by attack after electric shock therapy, by administering a therapeutically effective amount of at least one of zonisamide, salts thereof, solvates thereof and prodrugs thereof described herein.

“Cognitive impairment associated with chemotherapy” refers to cognitive impairment defined herein that is caused by or associated with chemotherapy. In other aspect, the present invention provides a method for alleviating (e.g., reducing or eliminating) cognitive impairment associated with chemotherapy, by administering a therapeutically effective amount of at least one of zonisamide, salts thereof, solvates thereof and prodrugs thereof described herein. In a preferred aspect, the present invention provides a method for treating cognitive impairment for a patient with breast cancer receiving chemotherapy, by administering a therapeutically effective amount of at least one of zonisamide, salts thereof, solvates thereof and prodrugs thereof described herein.

In each of the methods described herein, zonisamide, a salt thereof, a solvate thereof or a prodrug thereof described herein alleviates (e.g., reduces or eliminates) at least one (preferably, two, three or all) of the symptoms of a disease, a disorder or syndrome in treatment. Preferably, zonisamide, a salt thereof, a solvate thereof or a prodrug thereof alleviates symptoms of dementia and/or cognitive impairment.

In general, “treatment” refers to acquisition of a desired pharmacological effect and/or physiologic effect. These effects are prophylactic in terms of completely or partially preventing a disease and/or a symptom, and therapeutic in terms of partially or completely curing a disease and/or an adverse effect caused by a disease. As used herein, “treatment” includes any treatment of a disease of a patient, particularly human, including, for example, at least one of the following treatments (a) to (c):
(a) to prevent a disease or a symptom in a patient who is suspected of being predisposed to the disease or the symptom but not yet diagnosed to be so;
(b) to inhibit a symptom of a disease, that is, to inhibit or delay the progress of the symptom;
(c) to alleviate a symptom of a disease, that is, to reverse or eliminate the symptom of the disease, or to reverse the progress of the symptom.

For example, clinical symptoms of AD include progressive disorientation, amnesia and aphasia, which eventually cause incompetence, speech loss and akinesia. Examples of pathological signs of AD include neurofibrillary tangle, senile plaque and amyloid vascular disorder. “To prevent progression of AD” is interpreted as meaning to prevent onset or further progression of a clinical symptom and/or a pathological sign of AD. For example, progression of a clinical symptom or a pathological sign can be prevented for patients without the clinical symptom or the pathological sign of AD. In addition, a severer AD condition can be prevented for patients suffering from mild AD. “To delay the progression of AD” is interpreted as meaning to delay the onset of an AD-associated symptom and/or pathological sign, or to slow down the progression rate of AD as determined by the progression rate of the clinical symptom and pathological sign. “To reverse the progression of AD” is interpreted as meaning to alleviate the severity of a symptom of AD, that is, to alter the patient's symptom of AD from a severe to milder condition. In this case, alteration to a milder condition is indicated by reduction of the clinical symptom or the pathological sign.

AD diagnosis of a patient can be carried out by employing various known methods. Typically, clinical and pathological assessments are combined to diagnose AD. For example, progression or severity of AD can be assessed by using the Mini Mental State Examination (MMSE) (Mohs et al., (1996) Int Psychogeriatr 8:195-203), Alzheimer's Disease Assessment Scale-Cognitive Subscale (ADAS-cog) (Galasko et al., (1997) Alzheimer Dis Assoc Disord, 11 suppl 2:S33-9), Alzheimer's Disease Cooperative Study-Activities of Daily Living scale (ADCS-ADL) (McKhann et al., (1984) Neurology 34:939-944), the National Institute of Neurologic Communicative Disorders and the Stroke-Alzheimer's Disease and Related Disorders Association (NINCDS-ADRDA) criteria (Folstein et al., (1975) J Psychiatr Res 12:189-198, McKhann et al., (1984) Neurology 34:939-944). Furthermore, a method capable of assessing various areas in the brain to estimate frequency of senile plaque or neurofibrillary tangle may be used (Braak et al., (1991) Acta Neuropathol 82:239-259; Khachaturian (1985) Arch Neuro 42:1097-1105; Mirra et al., (1991) Neurology 41:479-486; and Mirra et al., (1993) Arch Pathol Lab Med 117:132-144).

2. Pharmaceutical composition

The present invention provides a method for treating dementia (preferably, AD or SDAT) or cognitive impairment described above. This method can be carried out by administering an effective amount of zonisamide to a patient in need of treatment of such a disease. The present invention comprises a pharmaceutical composition comprising zonisamide for treating a patient with dementia (preferably, AD or SDAT) or cognitive impairment described above. As used herein, the term “zonisamide” includes, unless otherwise stated, a salt of zonisamide, a solvate thereof and a prodrug thereof described below.

Zonisamide may be used in the same manner in its salt form. Salts refer to pharmaceutically acceptable salts known in the art, and not particularly limited as long as they form pharmaceutically acceptable salts with zonisamide. Specifically, examples include quatemized amine salts, alkali metal salts (e.g., sodium salt, potassium salt and lithium salt) and alkaline earth metal salts (e.g., magnesium salt and calcium salt).

Zonisamide may be produced according to a method described, for example, in Japanese Examined Application No. 60-33114, Japanese Examined Application No. 61-59288 and U.S. Pat. No. 4,172,896.

In addition, zonisamide or a salt thereof may be used in the same manner in its solvate form. According to the present invention, “solvate” is preferably a pharmacologically acceptable solvate. The pharmacologically acceptable solvate may be either a hydrate or a nonhydrate, preferably a hydrate. As a nonhydrate, alcohols (e.g., methanol, ethanol, n-propanol), dimethylformamide, dimethylsulfoxide (DMSO) or else can be used.

Moreover, zonisamide, a salt thereof or a solvate thereof may be used in the same manner in its prodrug form.

The term “prodrug” refers to “an active form of a drug” (i.e., the “drug” corresponding to the prodrug) that is chemically modified into an inactive substance for the purpose of improving bioavailability, alleviating side-effects or else, which is absorbed in the body and then metabolized into the active form to exert action. Thus, the term “prodrug” refers to any compound having a lower intrinsic activity than a corresponding “drug”, which, once administered into a biological system, generates the “drug” that provides specific activity through spontaneous chemical reaction or enzymatic catalysis or metabolic reaction. Examples of such prodrugs include compounds in which a sulfamoyl group or the like of zonisamide is acylated, alkylated, phosphorylated, borated, carbonated, esterified, amidated or urethanated. This exemplified group, however, is not comprehensive but merely a typical group of examples and those skilled in the art can prepare other known prodrugs from zonisamide according to a known method. Prodrugs produced from zonisamide are within the scope of the present invention.

Zonisamide, a salt thereof, a solvate thereof or a prodrug thereof may be administered either orally or parenterally (e.g., intravenous injection, intramuscular injection, subcutaneous administration, rectal administration, transdermal administration). The dose of zonisamide, a salt thereof, a solvate thereof or a prodrug thereof differs depending on various factors including, for example, the administration route, the type of the disease, the degree of the symptom, age, sex and weight of the patient, the type of the salt, specific type of the disease, pharmacological evidence such as pharmacokinetics and toxicological aspects, use of drug delivery system and whether or not it is administered in combination with other drugs. Generally, the dose for an adult (weighing 60 kg) is about 0.1 to 3000 mg/day, preferably about 1.0 to 600 mg/day, more preferably about 5 to 300 mg/day for oral administration, and about 0.01 to 500 mg/day, preferably about 0.1 to 100 mg/day, more preferably about 0.1 to 30 mg/day for injection, and may be administered at once or in several times. When administered to a child, the dose may possibly be lower than that for an adult. The actual method for administration may fluctuate widely and may depart from the preferred method described herein.

Zonisamide, a salt thereof, a solvate thereof or a prodrug thereof may be used alone or may be applied to a pharmaceutical composition formulated into an appropriate formulation using a pharmaceutical carrier by a conventional method selected depending on the administration route, as a therapeutic agent for dementia (preferably, AD or SDAT) or cognitive impairment described above. Specific examples of the pharmaceutical composition include oral agents such as a tablet, a powdered agent, subtle granules, granules, a coated tablet, a capsule, syrup and a lozenge, or parenteral agents such as an inhaler, a suppository, an injectable agent (including drips), an ointment, eye-drops, an eye ointment, nasal drops, ear drops, a patch, a lotion and a liposome agent. These pharmaceutical compositions are prepared according to a conventional method. A tablet, a powdered agent or the like that is commercially available as an antiepileptic agent may also be applied as a therapeutic agent for dementia (preferably, AD or SDAT) or cognitive impairment described above. Zonisamide is available as ExcegranT tablet (Dainippon Pharma Co., Ltd.), Excegranm powder (Dainippon Pharma Co., Ltd.) and as ZONEGRAN™ (Eisai Ink.) in the United States.

As the pharmaceutical carrier, for example, a substance is used that is conventionally used in the medical field and that does not react with zonisamide, a salt thereof, a solvate thereof or a prodrug thereof. The pharmaceutical carrier may be, for example, a generally used excipient, binder, disintegrating agent, lubricant, colorant, flavoring agent, and if necessary, a stabilizer, an emulsifier, an absorption promoter, a surfactant, a pH adjuster, an antiseptic, an antioxidant, a filler, a wetting agent, a surface active agent, a dispersing agent, a buffer, a preservative, a solubilizing agent, a soothing agent or the like, and can be formulated by a conventional method by blending components that are generally used as materials in medical products. Examples of such components available and nontoxic include animal and plant oils such as soybean oil, beef fat and synthesized glyceride; hydrocarbons such as liquid paraffin, squalane oil and solid paraffin; ester oils such as octyldodecyl myristate and isopropyl myristate; higher alcohols such as cetostearyl alcohol and behenyl alcohol; silicon resin; silicon oil; surfactants such as polyoxyethylene fatty acid ester, sorbitan fatty acid ester, glycerine fatty acid ester, polyoxyethylene sorbitan fatty acid ester, polyoxyethylene hydrogenated castor oil and polyoxyethylene-polyoxypropylene block copolymer; water-soluble polymers such as hydroxyethyl cellulose, polyacrylic acid, carboxy vinyl polymer, polyethylene glycol, polyvinylpyrrolidone and methylcellulose; lower alcohols such as ethanol and isopropanol; polyalcohols (polyols) such as glycerine, propylene glycol, dipropylene glycol, sorbitol and polyethylene glycol; sugars such as glucose and sucrose; inorganic powder such as silicic anhydride, magnesium aluminum silicate and aluminum silicate; inorganic salts such as sodium chloride and sodium phosphate; and purified water.

Examples of excipients include lactose, fructose, cornstarch, white sugar, glucose, mannitol, sorbit, crystalline cellulose, silicon dioxide and calcium sulfate; examples of binders include polyvinyl alcohol, polyvinyl ether, methylcellulose, ethyl cellulose, gum arabic, tragacanth, gelatin, shellac, hydroxypropyl methylcellulose, hydroxypropyl cellulose, polyvinylpyrrolidone, polypropylene glycol polyoxyethylene block copolymer and meglumine; examples of disintegrating agents include starch, agar, gelatin powder, crystalline cellulose, calcium carbonate, sodium hydrogen carbonate, calcium citrate, dextrin, pectine, carboxymethylcellulose calcium, carmellose sodium and carmellose calcium; examples of lubricants include magnesium stearate, talc, polyethylene glycol, silica and hydrogenated vegetable oil; examples of colorants include those that are acceptable as additives in pharmaceuticals; and examples of flavoring agents include cocoa powder, menthol, aromatic powder, mint oil, camphol and cinnamon powder. The above-mentioned components may be their salts or solvates thereof.

For example, an excipient, and if necessary, a binder, a disintegrating agent, a lubricant, a colorant, a flavoring agent or the like may be added to zonisamide, a salt thereof, a solvate thereof or a prodrug thereof prior to making an oral formulation such as a powdered agent, subtle granules, granules, a tablet, a coated tablet or a capsule by a conventional method.

A tablet may be coated by a well-known method using a coating agent such as camauba wax, hydroxypropyl methylcellulose, macrogol, hydroxypropyl methylphthalate, cellulose acetate phthalate, white sugar, titanic oxide, sorbitan fatty acid ester and calcium phosphate.

Specific examples of carriers used for producing a syrup product include sweetening agents such as white sugar, glucose and fructose, suspending agents such as gum arabic, tragacanth, carmellose sodium, methylcellulose, sodium alginate, crystalline cellulose and veegum and dispersing agents such as sorbitan fatty acid ester, sodium lauryl sulfate and polysorbate 80. If necessary, a flavoring agent, an aromatic agent, a preservative, a solubilizing agent, a stabilizer or the like may be added for producing a syrup product. The product may be in a dry syrup form for dissolving or suspending upon use.

Specific examples of suppository bases include those that are solid at room temperature and becomes liquid at body temperature, including, for example, appropriate nonstimulus excipients such as cacao butter, saturated fatty acid glycerine ester, polyethylene glycol, glycerogelatin and macrogol. For producing a suppository, a surfactant, a preservative or the like may be added if necessary.

An injectable agent is generally prepared by dissolving, for example, a salt of zonisamide into injectable distilled water, and if necessary, a solubilizing agent, a buffer, a pH adjuster, a tonicity agent, a soothing agent, a preservative, a stabilizer or the like may be added.

The production process for a topical agent is not particularly limited and may be produced according to a conventional method. For a base material used, various materials generally used in pharmaceuticals, quasi-drugs, cosmetics or the like may be used, including, for example, materials such as animal and plant oils, mineral oils, ester oils, waxes, higher alcohols, fatty acids, silicon oils, surfactants, phospholipids, alcohols, polyalcohols, water-soluble polymers, clay minerals and purified water. If necessary, a pH adjuster, an antioxidant, a chelating agent, antiseptics/fungicides, a colorant, a flavoring or the like may be added. In order to administer an inhaler by inhalation, zonisamide, a salt thereof, a solvate thereof or a prodrug thereof may be delivered by other suitable systems including an injector, a nebulizer, a pressurized package or aerosol spray. The pressurized package may contain an appropriate propellant. Furthermore, for inhaled administration, zonisamide, a salt thereof, a solvate thereof or a prodrug thereof may be administered in a form of dry powder composition or in a form of liquid spray. For local administration on the skin surface, zonisamide, a salt thereof, a solvate thereof or a prodrug thereof may be formulated into an ointment, a cream or a lotion, or as an active component for a transdermal patch. An ointment and cream may be formulated, for example, by adding an appropriate thickening and/or gelling agent to an aqueous or oily base. A lotion may be formulated by using an aqueous or oily base, and may generally contain one or more of an emulsifier, a stabilizer, a dispersing agent, a suspending agent, a thickening agent and/or a colorant.

Moreover, these pharmaceutical compositions may also contain other substances described below that are effective in treatment. If necessary, components such as a blood flow stimulator, a disinfectant, an antiphlogistic, a cellular stimulant, vitamins, amino acids, a moisturizing agent and a keratolytic agent may be blended. In these cases, the proportion of the active component to the carrier may vary within the range of 1 to 90% by weight.

In general, these pharmaceutical compositions may contain, as an active component, zonisamide, a salt thereof, a solvate thereof or a prodrug thereof in a proportion of 0.5% or higher (% by weight, the same applies hereinbelow), preferably 10 to 70%. When zonisamide, a salt thereof, a solvate thereof or a prodrug thereof is used for the treatments described above, it is purified to at least 90% or higher, preferably 95% or higher, more preferably 98% or higher, still more preferably 99% or higher.

3. Combination therapy

(1) Embodiments

The present invention relates to a method for treating dementia (preferably, AD or SDAT) or cognitive impairment described above by a combination therapy (hereinafter, referred to as a “combination therapy of the present invention”). The combination therapy of the present invention is provided as a concomitant administration of zonisamide, a salt thereof, a solvate thereof or a prodrug thereof with a drug used for or a drug under development for the treatment of dementia (preferably, AD or SDAT) or cognitive impairment described above (preferably, cholinesterase inhibitor (ChEI), NMDA receptor antagonist, AMPA receptor antagonist).

In another embodiment of the invention, a combination therapy of the invention is provided as a pharmaceutical composition (blended agent) containing zonisamide, a salt thereof, a solvate thereof or a prodrug thereof concomitantly with a drug used for or a drug under development for treating dementia (preferably, AD or SDAT) or cognitive impairment described above (preferably, CHEI, NMDA receptor antagonist, AMPA receptor antagonist).

(2) Pharmaceutical composition (blended agent)

(i) The present invention provides a pharmaceutical composition (blended agent) containing zonisamide, a salt thereof, a solvate thereof or a prodrug thereof concomitantly with a drug used for or a drug under development for treating dementia (preferably, AD or SDAT) or cognitive impairment described above. Preferably, a pharmaceutical composition (blended agent) is provided which contains zonisamide, a salt thereof, a solvate thereof or a prodrug thereof and at least one selected from the group consisting of ChEIs, NMDA receptor antagonists and AMPA receptor antagonists.

(3) Drug used for or drug under development for treating dementia (preferably, AD or SDAT) or cognitive impairment described above

(i) ChEI

ChEI is a compound for inhibiting ChE, i.e., a compound that reversibly or irreversibly inhibits ChE activity. ChEs include ACHE, butyryl cholinesterase or the like. For example, preferable features of the ChEI of the present invention include that is has higher selectivity to AChE than to butyryl cholinesterase, that it is capable of passing through a blood-brain barrier, and that it does not cause severe side-effects in a dose required for treatment.

For a pharmaceutical composition of the present invention, compounds preferable to be used concomitantly and blended with zonisamide, a salt thereof, a solvate thereof or a prodrug thereof include at least one selected from the group consisting of ChEIs, salts thereof and solvates thereof, particularly at least one selected from the group consisting of AChEIs, salts thereof and solvates thereof. A salt of ChEI refers to pharmaceutically acceptable salts known in the art, and are not particularly limited as long as they form pharmaceutically acceptable salts with ChEI. Specifically, examples include halogenated hydrohalic acid salts (e.g., hydrofluorate, hydrochloride, hydrobromide and hydroiodide), inorganic acid salts (e.g., sulfate, nitrate, perchlorate, phosphate, carbonate and bicarbonate), organic carboxylates (e.g., acetate, oxalate, maleate, tartrate, fumarate and citrate), organic sulfonates (e.g., methanesulfonate, trifluoromethanesulfonate, ethanesulfonate, benzenesulfonate, toluenesulfonate and camphorsulfonate), amino acid salts (e.g., aspartate and glutamate), quaternized amine salts, alkali metal salts (e.g., sodium salt and potassium salt) and alkaline earth metal salts (e.g., magnesium salt and calcium salt).

Examples of ChEIs used with the present invention include donepezil (ARICEPT™), galanthamine (Reminyl™), Tacrine (Cognex™), rivastigmine (Exelon™), zifrosilone (U.S. Pat. No. 5,693,668), physostigmine (Synapton), ipidacrine (U.S. Pat. No. 4,550,113), quilostigmine, Metrifonate (Promem) (U.S. Pat. No. 4,950,658), eptastigmine, velnacrine, tolserine, cymserine (U.S. Pat. No. 6,410,747), Mestinon, icopezil (U.S. Pat. No. 5,750,542), TAK-147 (J. Med. Chem., 37(15), 2292-2299, 1994, Japanese Patent No. 2650537, U.S. Pat. No. 5,273,974), huperzine A (Drugs Fut., 24, 647-663, 1999), stacofylline (U.S. Pat. No. 4,599,338), thiatolserine, Neostigmine, eseroline or thiacymserine, 8-[3-[1-[(3-fluorophenyl)methyl]-4-piperidinyl]-1-oxopropyl]-1,2,5,6-tetrahydro-4H-pyrro [3,2,1-ij]quinoline-4-one (Japanese Patent No. 3512786), Phenserine, ZT-1 or enantiomers of the compounds exemplified above, derivatives thereof or prodrugs thereof.

For example, donepezil hydrochloride may be produced readily by typical methods disclosed in Japanese Laid-Open Patent Application No. 1-79151, Japanese Patent No. 2578475, Japanese Patent No. 2733203, Japanese Patent No. 3078244 or U.S. Pat. No. 4,895,841. For example, galanthamine and derivatives thereof are described in U.S. Pat. No. 4,663,318, WO88/08708, WO97/03987, U.S. Pat. No. 6,316,439, U.S. Pat. No. 6,323,195 and U.S. Pat. No. 6,323,196. For example, Tacrine and derivatives thereof are described in U.S. Pat. No. 4,631,286, U.S. Pat. No. 4,695,573, U.S. Pat. No. 4,754,050, WO88/02256, U.S. Pat. No. 4,835,275, U.S. Pat. No. 4,839,364, U.S. Pat. No. 4,999,430 and WO97/21681. For example, rivastigmine and derivatives thereof are described in European patent No.193926, WO98/26775 and WO98/27055. Furthermore, an example of ChEI used with the present invention includes ChEI described in WO00/18391.

Examples of prodrugs include various prodrugs where an amino group, a hydroxyl group, a carboxyl group or else of the compounds exemplified above is acylated, alkylated, phosphorylated, borated, carbonated, esterified, amidated or urethanated. This group of examples, however, is not comprehensive but merely a typical group of examples and those skilled in the art may prepare other known prodrugs from the compounds exemplified above according to a known method. A prodrug produced from a compound exemplified above is within the scope of the present invention.

(ii) NMDA receptor antagonist

NMDA receptor antagonist according to the present invention refers to at least one selected from the group consisting of: compounds that bind with a NMDA receptor and inhibit the flnctions thereof; salts thereof; and solvates thereof. Examples of the NMDA receptor antagonists of the present invention include memantine (3,5-Dimethyl-adamantan-1-ylamine; CAS#L19982-08-2), derivatives thereof, salts thereof, solvates thereof or prodrugs thereof. The salts may be those exemplified in “(i) ChEI” above, and preferably is hydrochloride. Examples of prodrugs include various prodrugs where an amino group, a hydroxyl group, a carboxyl group or else of the compounds exemplified above is acylated, alkylated, phosphorylated, borated, carbonated, esterified, amidated or urethanated. This group of examples, however, is not comprehensive but merely a typical group of examples, and those skilled in the art may prepare other known prodrugs from the compounds exemplified above according to a known method. A prodrug produced from a compound exemplified above is within the scope of the present invention. Memantine and derivatives thereof, and their production methods are described in Japanese Patent No. 2821233, Curr Opin Investig Drugs. 2002 May; 3(5): 798-806.

(iii) AMPA receptor antagonist

The AMPA receptor antagonist used with the present invention refers to at least one selected from the group consisting of: compounds that bind with a NMDA receptor and inhibit the flnctions thereof; salts thereof; and solvates thereof. Examples of AMPA receptor antagonists of the present invention include Talampanel (LY300164; (R)-(−)-1-(4-Aminophenyl)-3-acetyl-4-methyl-7,8-methylenedioxy-3,4-dihydro-5H-2,3-benzodiazepine; CAS#161832-65-1), derivatives thereof, salts thereof, solvates thereof or prodrugs thereof. The salts may be those exemplified in “(i) ChEI” above. Examples of prodrugs include various prodrugs where an amino group, a hydroxyl group, a carboxyl group or else of the compounds exemplified above is acylated, alkylated, phosphorylated, borated, carbonated, esterified, amidated or urethanated. This group of examples, however, is not comprehensive but merely a typical group of examples, and those skilled in the art may prepare other known prodrugs from the compounds exemplified above according to a known method. A prodrug produced from a compound exemplified above is within the scope of the present invention. A method for producing talampanel is described in J. Chem. Soc. Perkin Trans. I, 1995, p. 1423.

(iv) Other drugs

According to the present invention, examples of drugs under development for treating dementia (preferably, AD or SDAT) or cognitive impairment described above include Xaliproden (SR 57746), Alzhemedm, Anapsos, PTI 00703, Cereact (ONO 2506), R-flurbiprofen, SR 57667, PYM 50028, C 9136, FK949 (quetiapine fumarate), FK962, Estradex, Bacosides A&B, LY450139, AAB001, Nitroflurbiprofen, PPI 1019, iAbeta5, TTP488, HF 0220, HF 0420, C 7617, C 9138, T-588, S-8510, MKC-231, MND-21, 737552 (benzodiazepine partial inverse agonist), 742457 (5HT6 antagonist), AAV-NGF (CERE-110), Ampalex, APAN, Avandia, CPI-1189, DP 543, MEM 1003, MPC-7869, NS 2330, R1485, R1500, R1533, Zyprexa or prodrugs thereof, and combinations with zonisamide, a salt thereof, a solvate thereof or a prodrug thereof are within the scope of the present invention. Examples of prodrugs of the drugs under development described above include various prodrugs where an amino group, a hydroxyl group, a carboxyl group or else of the compounds exemplified above is acylated, alkylated, phosphorylated, borated, carbonated, esterified, amidated or urethanated. This group of examples, however, is not comprehensive but merely a typical group of examples, and those skilled in the art may prepare other known prodrugs from the compounds exemplified above according to a known method. A prodrug produced from a compound exemplified above is within the scope of the present invention.

(4) Administration form

Concomitant use of zonisamide, a salt thereof, a solvate thereof or a prodrug thereof with a drug used for or a drug under development for treating dementia (preferably, AD or SDAT) or cognitive impairment described above (preferably, cholinesterase inhibitor (ChEI), NMDA receptor antagonist or AMPA receptor antagonist) is useful in treating dementia (preferably, AD or SDAT) or cognitive impairment described above. In another embodiment of the present invention, a pharmaceutical composition (blended agent) containing zonisamide, a salt thereof, a solvate thereof or a prodrug thereof with a drug used for or a drug under development for treating dementia (preferably, AD or SDAT) or cognitive impairment described above (preferably, ChEI, NMDA receptor antagonist or AMPA receptor antagonist) is useful in treating dementia (preferably, AD or SDAT) or cognitive impairment described above.

In a combination therapy of the present invention, the components for concomitant use may be given at their effective amounts at the same time or with time interval, or they may be given at their effective amounts as pharmaceutical compositions formulated according to a conventional method at the same time or with time interval. Alternatively, in a combination therapy of the present invention, the components for concomitant use may be given at their effective amounts by formulation in which they are directly blended, or they may be given at their effective amounts by formulation in which they are formulated to some extent and then blended. Formulation can be carried out by those skilled in the art based on a conventional technique (see “2. Pharmaceutical composition” above).

An administration form of the pharmaceutical composition used in the combination therapy of the present invention is not particularly limited and may be administered orally or parenterally (see “2. Pharmaceutical composition” above). Administration forms or doses of the components to be used concomitantly or blended may differ upon concomitant use or upon blending.

For administration forms and doses of zonisamide, a salt thereof, a solvate thereof or a prodrug thereof, see “2. pharmaceutical composition” above.

As an oral administration form of ChEI, a tablet, subtle granules and the like are commercially available. For example, a subtle granule of donepezil hydrochloride is available under the trade name of ARICEPT granule (Eisai Co., Ltd.) and a tablet is available under the trade name of ARICEPT tablet (Eisai Co., Ltd.). When administration via transdermal absorption with a patch is employed, ChEI is preferably selected which does not form a salt, i.e., which is a free form. For local administration on skin surface, ChEI may be formulated as an ointment, a cream or a lotion, or as an active component for a transdermal patch. An ointment and a cream may be formulated, for example, by adding an appropriate thickening and/or gelling agent to an aqueous or oily base. A lotion may be formulated by using an aqueous or oily base, and may contain generally one or more of an emulsifier, a stabilizer, a dispersing agent, a suspending agent, a thickening agent and/or a colorant. ChEI may also be administered by ion transfer therapy.

A dose of ChEI for an oral administration is about 0.001 to 1000 mg/day, preferably about 0.01 to 500 mg/day, more preferably about 0.1 to 300 mg/day for an adult (weighing 60 kg). For example, a dose of donepezil hydrochloride is preferably about 0.1 to 300 mg/day, more preferably about 0.1 to 100 mg/day, more preferably about 1.0 to 50 mg/day for an adult (weighing 60 kg). In a preferred aspect of donepezil hydrochloride, 5 mg or 10 mg tablets of donepezil hydrochloride commercially available as ARICEPT (Eisai Co., Ltd.) may be administered. Tablets can be administered for 1 to about 4 times/day. In a preferred aspect, a 5 mg or a 10 mg ARICEPT tablet (Eisai Co., Ltd.) is administered once a day. Those skilled in the art will appreciate that when administering donepezil hydrochloride to a child, the dose may possibly be lower than that for an adult, and in a preferred aspect, donepezil hydrochloride may be administered to a child at about 0.5 to 10 mg/day, preferably about 1.0 to 3 mg/day. Preferably, for Tacrine, about 0.1 to 300 mg/day, preferably about 40 to 120 mg/day; for rivastigmine, about 0.1 to 300 mg/day, preferably about 3.0 to 12 mg/day; for galanthamine, about 0.1 to 300 mg/day, preferably about 16 to 32 mg/day is administered to an adult (weighing 60 kg). When these are administered to a child, the dose may possibly be lower than that for an adult.

For parenteral administration, a preferable dose of ChEI for patch application is about 5.0 to 50 mg/day, preferably about 10 to 20 mg/day for an adult (weighing 60 kg). An injectable agent may be produced by dissolving or suspending in a pharmaceutical carrier such as saline or commercially available injectable distilled water to a concentration of about 0.1 μg/ml carrier to about 10 mg/ml carrier. The injectable agent produced like this can be administered at about 0.01 to 5.0 mg/day, more preferably about 0.1 to 1.0 mg/day for 1 to 3 times/day for an adult (weighing 60 kg). When administering to a child, the dose may possibly be lower than that for an adult.

The administration forms and the doses of NMDA receptor antagonists (e.g., memantine), AMPA receptor antagonists (e.g., talampanel) and other drugs described above depend on the subject to be administered, administration route, property of the formulation, condition of the patient and physician's judgment. An oral administration form of an NMDA receptor antagonist is commercially available as a tablet. For example, a memantine hydrochloride tablet is available under the trade name of Namenda (Forest Laboratories). Although a preferred treatment dose of memantine for oral administration is about 5.0 to 35 mg/day for an adult (weighing 60 kg), use of memantine at a dose of about 100 to 500 mg/day for treatment is well acceptable. Talampanel may be used at about 20 to 70 mg, preferably about 20 to 50 mg for an adult (weighing 60 kg) for 2 to 4 times/day, preferably 3 times/day. An injectable agent may be produced by dissolving or suspending in a pharmaceutical carrier such as saline or commercially available injectable distilled water to a concentration of about 0.1 μg/ml carrier to about 10 mg/ml carrier. A dose of the injectable agent produced like this can be administered at about 0.01 to 5.0 mg/day, more preferably about 0.1 to 1.0 mg/day for 1 to 3 times/day for an adult (weighing 60 kg). A dose of an NMDA receptor antagonist or an AMPA receptor antagonist is not particularly limited to those described above, and the dose may vary depending on a compound, a salt thereof or a solvate thereof to be administered and the efficiency of the administration route. For example, an oral administration is expected to require a higher dose than that for an intravenous injection. Such fluctuation in the dose level may be regulated by using a standard empirical optimization procedure well known in the art. When administering to a child, the dose may possibly be lower than that administered to an adult.

The dose upon concomitant use or blending described above may appropriately be selected from the doses mentioned above.

### EXAMPLES

Hereinafter, the present invention will be described in more detail by way of examples. The present invention, however, is not limited to these examples, which are described for providing those skilled in the art with a complete disclosure. Moreover, description of these experiments does not mean or suggest that they are all and the only experiments conducted. Although efforts have been made to ensure accuracy of the numerical values used herein (e.g., amount, temperature, concentration, etc.), some degree of experimental error and deviation are within consideration, and may vary within a range that does not depart from the scope the present invention.

### Example 1

### Subjects

Male and female patients aging from 60 to 85 suffering from mild to moderate AD who fulfill the NINCDS-ADRDA criteria will be recruited, and 50 to 100 patients will be registered as a zonisamide treatment group for the main test. Physicians judge the patients who will be applied for the main test based on the NINCDS-ADRDA criteria before administration. The physicians also will diagnose patients' clinical signs such as disorientation, impaired memory, impaired judgment, impaired intellect, anxiety, depression, agitation and deterioration in activity in daily life and will judge based on all information available at the point of analysis such as report from family members or care attendants, and period of care. AD subjects will be attended by care attendants who will make sure of compliance with dosing regimen, hospital visit and procedure for this test. Patients suffering from other disease with a medically severe symptom, patients with any contraindication or patients who regularly will use zonisamide will be eliminated. Patients receiving treatment with AChEI for AD will not be eliminated if they will be administered with a stable dose for at least several weeks (preferably, 4 weeks).

Drug

To AD patients who will fulfill the NINCDS-ADRDA criteria, zonisamide will be administered at an amount formulated in a carrier appropriate for the selected dosing regimen. The patients will start with zonisamide treatment protocol. An amount of zonisamide in a range of about 10 to 100 mg/day, for example, 40 mg/day will be prescribed in 1 to 3 doses. During the test period, administration will be repeated daily. Starting from day 0, a test for assessment scale will be conducted regularly, for example, once a month.

Assessment

The zonisamide treatment group will be evaluated for one or several assessment items including, for example, a test for cognitive and memory function, Aβ present in cerebrospinal cord fluid or plasma, Aβ deposition in the brain, brain amyloid plaque, or a volume of a brain or hippocampus. In the zonisamide treatment group, progression of the disease will be inhibited or delayed or a symptom of the disease will be alleviated as compared to the untreated control group. Specifically, results will be obtained where the degree or period of patients' disorientation, impaired memory, impaired judgment and impaired intellect will be reduced, patients' anxiety, depression, agitation, frustration or fecklessness will be reduced, or patients start to feel stable, will become socially active, or will become to have improved language ability or comprehension.

1-2. (canceled)

3. A method for treating dementia or cognitive impairment, comprising administering an effective amount of zonisamide, a salt thereof, a solvate thereof or a prodrug thereof to a patient in need of treatment for these diseases.

4. A method according to claim 3, wherein the dementia or cognitive impairment is one selected from the group consisting of:
(a) Alzheimer's disease, senile dementia of the Alzheimer's type, dementia associated with vascular disease, dementia caused by vascular disease, dementia associated with Parkinson's disease, dementia associated with Huntington's disease, dementia associated with alcohol addiction, Lewy body dementia and AIDS dementia;
(b) mild cognitive impairment and age-related impaired memory;
(c) dementia and/or cognitive impairment associated with epilepsy, dementia and/or cognitive impairment associated with cerebral tumor, dementia and/or cognitive impairment associated with brain injury, dementia and/or cognitive impairment associated with multiple sclerosis, dementia and/or cognitive impairment associated with Down's syndrome, dementia and/or cognitive impairment associated with Rett's syndrome, dementia and/or cognitive impairment associated with progressive supranuclear palsy, dementia and/or cognitive impairment associated with frontal lobe syndrome, and dementia and/or cognitive impairment associated with neurologic and/or psychiatric conditiones; and
(d) cognitive impairment associated with traumatic brain injury, cognitive impairment associated with aftereffects of coronary-artery bypass surgery, cognitive impairment associated with electric shock therapy and cognitive impairment associated with chemotherapy.

5. A method according to claim 3 or 4, wherein the patient is human.

6. A pharmaceutical composition comprising zonisamide, a salt thereof, a solvate thereof or a prodrug thereof together with at least one selected from the group consisting of cholinesterase inhibitors, NMDA receptor antagonists and AMPA receptor antagonists.

7. A pharmaceutical composition according to claim 6, wherein the cholinesterase inhibitor is donepezil or a salt thereof.

8. A pharmaceutical composition according to claim 6, wherein the cholinesterase inhibitor is donepezil hydrochloride.

9. A pharmaceutical composition according to claim 6, wherein the cholinesterase inhibitor is galanthamine or a salt thereof.

10. A pharmaceutical composition according to claim 6, wherein the NMDA receptor antagonist is memantine.

11. A pharmaceutical composition according to claim 6, wherein the AMPA receptor antagonist is talampanel.

12-13. (canceled)

14. A method for treating dementia or cognitive impairment, comprising administering zonisamide, a salt thereof, a solvate thereof or a prodrug thereof together with at least one selected from the group consisting of cholinesterase inhibitors, NMDA receptor antagonists and AMPA receptor antagonists to a patient in need of treatment for these diseases.

15. A method according to claim 14, wherein the cholinesterase inhibitor is donepezil or a salt thereof.

16. A method according to claim 14, wherein the cholinesterase inhibitor is donepezil hydrochloride.

17. A method according to claim 14, wherein the cholinesterase inhibitor is galanthamine or a salt thereof.

18. A method according to claim 14, wherein the NMDA receptor antagonist is memantine.

19. A method according to claim 14, wherein the AMPA receptor antagonist is talampanel.

20. The method according to claim 14, wherein the dementia or cognitive impairment is one selected from the group consisting of:
(a) Alzheimer's disease, senile dementia of the Alzheimer's type, dementia associated with vascular disease, dementia caused by vascular disease, dementia associated with Parkinson's disease, dementia associated with Huntington's disease, dementia associated with alcohol addiction, Lewy body dementia and AIDS dementia;
(b) mild cognitive impairment and age-related impaired memory;
(c) dementia and/or cognitive impairment associated with epilepsy, dementia and/or cognitive impairment associated with cerebral tumor, dementia and/or cognitive impairment associated with brain injury, dementia and/or cognitive impairment associated with multiple sclerosis, dementia and/or cognitive impairment associated with Down's syndrome, dementia and/or cognitive impairment associated with Rett's syndrome, dementia and/or cognitive impairment associated with progressive supranuclear palsy, dementia and/or cognitive impairment associated with frontal lobe syndrome, and dementia and/or cognitive impairment associated with neurologic and/or psychiatric conditiones; and
(d) cognitive impairment associated with traumatic brain injury, cognitive impairment associated with aftereffects of coronary-artery bypass surgery, cognitive impairment associated with electric shock therapy and cognitive impairment associated with chemotherapy.

21. A method according to any one of claims 14 to 20, wherein the patient is human.
